SEQUENCE LISTING (2 sequences)

SEQ ID NO: 1 - protein, 20 aa, Homo sapiens
KSRITSEGEYIPLDQIDINV

SEQ ID NO: 2 - protein, 7 aa, Homo sapiens
FCYENEV

(19) United States
(12) Patent Application Publication
(10) Pub. No.: US 2020/0405885 A1
(43) Pub. Date: Dec. 31, 2020

(54) MAGNETIC RESONANCE IMAGING (MRI) CONTRAST AGENTS AND USE THEREOF

(71) Applicant: TECHNION RESEARCH & DEVELOPMENT FOUNDATION LTD., Haifa (IL)

(72) Inventor: Shai BERLIN, Binyamina (IL)

(21) Appl. No.: 16/629,689

(22) PCT Filed: Jul. 10, 2018
(86) PCT No.: PCT/IL2018/050752

§ 371 Date: Jan. 9, 2020

Related U.S. Application Data
(60) Provisional application No. 62/530,315, filed on Jul. 10, 2017.
(60) Provisional application No. 62/638,356, filed on Mar. 5, 2018.

(51) Int. Cl. A61K 49/00; A61K 49/10; A61K 49/14

(52) U.S. Cl. CPC A61K 49/0002; A61K 49/108; A61K 49/0052; A61K 48/00; A61K 49/0056; A61K 49/14; A61K 49/0047; A61K 49/0041

(57) ABSTRACT

A hybrid molecule comprising at least one contrast agent, and at least one substrate of a self-labeling enzyme, and optionally a fluorescent moiety is provided. Compositions comprising same and use thereof, are also provided.

### CROSS REFERENCE TO RELATED APPLICATIONS

This application claims the benefit of priority to U.S. Provisional Patent Application No. 62/530,315 filed Jul. 10, 2017, and U.S. Provisional Patent Application No. 62/638,356 filed Mar. 5, 2018 the contents of which are incorporated herein by reference in their entirety.

### FIELD OF THE INVENTION

The present invention is in the field of clinical anatomical imaging.

### BACKGROUND OF THE INVENTION

X-ray computed tomography (CT), magnetic resonance imaging (MRI) and light microscopy are used as separate modalities for diagnostic imaging. However, each approach has several limitations. For example, light-microscopy is invasive, radiative (i.e., may damage biological tissues) and displays poor tissue penetration. CT offers very limited tissue contrast (especially for soft tissues as the brain) and poor sensibility from a targeted molecular imaging perspective. MRI, on the other hand, is non-invasive and non-damaging, though is limited by its spatial and temporal resolutions, contrast, and cellular specificity. Methods for increasing contrast for these imaging techniques typically include the addition of contrast-enhancing agents, such as Gadolinium (Gd), Manganese (Mn) and iron (Fe) for MRI detection and Iodine (I) and Barium (Ba) for CT detection. However, contrast agents cannot differentiate between cell types or target regions (e.g., regions of the brain).

Another limitation when using contrast agents is obtaining a sufficiently high local concentration at desired regions, since these agents cannot be targeted. This requires using high doses of the agents, which may have cytotoxic effects. Lastly, contrast agents are readily cleared from the tissue, thus limiting the time-window in which patients can be imaged by MRI. Due to these limitations, despite the broad application of MRI in the clinic for assessing anatomical features of healthy or diseased tissue, this technique does not serve as a deterministic diagnostic tool for many diseases or disorders, including but not limited to neuropsychiatric or neurodegenerative diseases these conditions do not necessarily display noticeable neuroanatomical changes; especially at early or intermediate stages of the diseases. As such, ways of improving the diagnostic capabilities of these imaging techniques are greatly needed.

### SUMMARY OF THE INVENTION

The present invention provides hybrid molecules comprising a contrast agent a fluorochrome and a substrate of a self-labeling enzyme for use in cell-specific imaging. Methods of performing said imaging as well as kits comprising the hybrid molecule are also provided.

According to a first aspect, there is provided a hybrid molecule comprising at least one contrast agent and at least one substrate of a self-labeling enzyme.

According to some embodiments, the hybrid molecule of the invention further comprises at least one fluorescent moiety.

According to some embodiments, the contrast agent is a radiocontrast agent. According to some embodiments, the contrast agent is selected from the group consisting of a T1-class and a T2-class MRI contrast agent. According to some embodiments, the at least one contrast agent is iodine based.

According to some embodiments, the at least one contrast agent comprises a metal selected from the group consisting of: a superparamagnetic metal, a paramagnetic metal, a diamagnetic metal, a ferromagnetic metal, or any combination thereof. According to some embodiments, the at least one paramagnetic metal is selected from the group consisting of Barium (Ba), Tantalum (Ta), Tungsten (W), Dysprosium (Dy), Platinum (Pt), Gadolinium (Gd), and Manganese (Mn). According to some embodiments, the at least one diamagnetic metal is selected from the group consisting of Bismuth (Bi) and Gold (Au). According to some embodiments, the ferromagnetic metal is iron (Fe).

According to some embodiments, the self-labeling enzyme is capable of covalently binding to the substrate. According to some embodiments, the self-labeling enzyme is selected from the group consisting of: SNAP, CLIP and Halo.

According to some embodiments, the substrate is selected from the group consisting of: O6-benzylguanine derivatives, O2-benzylcytosine derivatives, and chloroalkane derivatives.

According to some embodiments, the fluorescent moiety is capable of emitting UV, visible, or near infrared light. According to some embodiments, the fluorescent moiety is selected from the group consisting of: green fluorescent protein (GFP) and Tomato.

According to some embodiments, the molecule has the general Formula:

(R)ₙ—(F)ₘ—(S)ₚ,
wherein:
R represents the contrast agent;
F represents the fluorescent moiety, and
S represents the substrate,
wherein n and p are each, independently, an integer from 1 to 5 and m is an integer from 0-5.

According to some embodiments, the hybrid molecule of the invention is for use in a cell-specific imaging assay.

According to another aspect, there is provided a composition comprising a hybrid molecule of the invention and a pharmaceutically acceptable carrier, excipient or adjuvant.

According to some embodiments, the composition of the invention comprises at least a first and a second hybrid molecule, wherein the substrate of the first hybrid molecule differs from the substrate of the second hybrid molecule, and at least one of the contrast agent and the fluorescent moiety of the first hybrid molecule differs from the contrast agent and the fluorescent moiety of the second hybrid molecule.

According to another aspect, there is provided a cell-specific imaging method, the method comprising contacting one or more cells with a composition of the invention wherein at least one cell of the one or more cells expresses one or more self-labeling enzymes capable of binding to the substrate, thereby imaging a specific cell.

According to some embodiments, the one or more cells express the one or more self-labeling enzymes on the at least one cell's surface.

According to some embodiments, the one or more cells are in the form of a tissue.

According to some embodiments, the contacting is performed ex-vivo or in-vivo.

According to some embodiments, the at least one cell is selected from a cancerous cell, a neuronal cell, and an immune cell.

According to some embodiments, the method of the invention comprises a preliminary step of expressing within the at least one cell the self-labeling enzymes capable of binding to the substrate. According to some embodiments, the expressing comprises introducing into the at least one specific cell one or more nucleic acid molecules comprising a polynucleotide sequence encoding the one or more self-labeling enzymes. According to some embodiments, the polynucleotide sequence encoding the one or more self-labeling enzymes is operatively linked to a cell-specific promoter.

According to some embodiments, the method of the invention further comprises applying a magnetic field, X-radiation, UV-vis light or any combination thereof, to the contacted cells.

According to some embodiments, the method of the invention further comprises measuring or detecting the contrast agent, the fluorescent moiety or both.

According to another aspect, there is provided a kit comprising:
(a) at least one hybrid molecule of the invention; and
(b) one or more nucleic acid constructs comprising a polynucleotide sequence encoding one or more self-labeling enzymes.

According to some embodiments, the polynucleotide sequence encoding one or more self-labeling enzyme is operatively linked to a cell-specific promoter.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The present invention is illustrated by way of example, and not by way of limitation, in the figures of the accompanying drawings and in which like reference numerals refer to similar elements and in which:

FIGS. 1A-C present Dual Magnetic and Fluorescent substrates for labeling selected neuronal populations to be imaged by MRI and Light microscopy. (1A) A non-limiting schematic drawing of the Magnetic (circle)-Fluorescent (star)-Substrate (triangle or trapezoid) contrast agents (MFS). Top molecule is a Gadolinium-based MFS (Gd—FS), whereas bottom molecule depicts an Iron-based MFS (Fe—FS). Each molecule may contain a different fluorescent molecule (e.g., FITC, Alexa, Atto, rhodamine and derivatives thereof including bit not limited to rhodamine-silicone derivatives, etc.). (1B) The substrate of the MFS binds irreversibly to genetically-encoded enzymes, e.g., SNAP and CLIP-tag (circles with open cavities), designed to be expressed at the membrane of cells, along with a fluorescent tag using cell specific promoters. When the SNAP- and CLIP-tags are at the membrane of cells (here neurons), they bind to the applied MFS, thereby immobilizing it. If two MFS are used in the same preparation, different cells will immobilize different MFS. Each contrast agent yields a different MRI signal (T1, T2, dashed circle and ellipse) that could be isolated during the MRI scan and also each MFS agent has a different fluorescent reporter. Each cell, with a unique combination of MFS and enzyme, will exhibit two colors of fluorescence (assessed by light microscopy) along with distinct MRI signatures. (1C) Example, but not limited to, of a brain depicting noticeable and distinct labeling of two distinct cellular population in the brain exhibit two distinct labeling schemes assessed by MRI (white and black).

FIGS. 2A-B present chemical structures of synthesized MFS-agents; (2A) substrate bound to a tetraazacyclododecane-1,4,7,10-tetraacetic acid or gadoteric acid (DOTA)-Gd³+1 (2B) and substrate bound to a fluorescent moleculeTexas Red and DOTA-Gd³+2.

FIGS. 3A-B present liquid Chromatography-Mass Spectrometry (LCMS) chromatograms of compound 1.

FIGS. 4A-C present the characterization of the MFS-agent 2 using different imaging modalities; (4A) Phantom T1 MRI-measurement (Bruker, ASPECT 1 T MRI scanner, USA) of control (0 mM) and MFS-agent (0.1 mM dissolved in water), (4B) Emission spectra of the MFS measured by confocal microscope (LSM-880, Zeiss, Germany) with 561 nm laser excitation (peak emission (nm): 617), (4C) and Phantom measurement in X-ray micro-Computed Tomography scanner (Bruker, Skyscan micro-CT, USA) of control (0 mM) and MFS-agent (0.1 mM dissolved in water).

FIG. 5 presents schematic illustration of engineered eTags; SNAP tag 1; CLIP tag 7; TM-2; Td-Tomato-3; GFP 4; FCYENEV (ER export motif) 5; ERX-golgi export and membrane trafficking motif 6; stop codon*.

FIGS. 6A-C present micrographs of (6A) SNAP-tdTomato and (6B) CLIP-GFP expressed in HeLa cells (Group III). Both constructs display robust expression (excitation 488 and 561 nm, respectively). (6C) A line graph showing an analysis of membrane localization. To assess membrane localization, fluorescence was collected over the length of a line spanning 10 μm across the membrane. The line was drawn so that membrane fluorescence would peak at 5 μm. Both plots show that fluorescence persists in the intracellular, demonstrating large intracellular retention of the constructs. Each plot is the summary of 25 cells. Plots are depicted as mean±SEM.

FIGS. 7A-C present micrographs of (7A) Poly-SNAP(x3)-tdTomato and (7B) poly-CLIP(x3)-GFP expressed in HeLa cells (Group VII). Both constructs display robust expression (excitation 488 and 561 nm, respectively). (7C) A line graph showing analysis of membrane localization (see FIG. 6C for details). SNAP(x3)-tdTomato shows very good membrane localization, whereas CLIP (x3)-GFP shows both membrane and intracellular expression in most cells. Each plot is the summary of 25 cells. Plots are depicted as mean±SEM.

FIG. 8 presents membrane targeted eTags; Inset-micrograph of a HeLa cells expressing SNAP(x3)-tdTomato labeled with Alexa488 conjugated to benzylguanine (BG). Plot shows the colocalization of both the fluorescent protein (Tdtomato) and of the dye (Alexa488) at the membrane of the cell. This demonstrates that the eTags are functional (i.e., conjugate BG substrate).

FIG. 9A-B presents the permeability of hybrid molecules with rhodamine content. (9A) A micrograph of HeLa cells incubated without (top left) or with (bottom left) MFS agents. Bottom left shows intracellular accumulation of the agents (see as red fluorescence) after washing away excess agents. Right image shows a compound image of transmitted light and fluorescence images, showing that the agents (red fluorescence noted by black arrowheads) accumulate in cytoplasm and not nucleus (denoted by dashed white lines in several cells). Grey filled circle denotes regions from where background was collected. (9B) Plots displaying fluorescence inside cells (black plot in accordance with black arrowheads in (9A)) as compared to background (collected from regions as noted in 9A).

### DETAILED DESCRIPTION OF THE INVENTION

According to some embodiments, the present invention provides a hybrid molecule comprising at least one contrast agent and at least one substrate of a self-labeling enzyme.

In some embodiments, the contrast agent is a radiocontrast agent.

The phrase “radiocontrast agent” refers to a group of contrast media typically used to improve the visibility of internal body structures in X-ray based imaging techniques.

The phrase “X-ray based imaging techniques” refers to a group of medical imaging technics that make use of X-radiation such as computed tomography where tomographic images or slices of specific areas of the body are obtained from a large series of two-dimensional X-ray images taken in different directions.

In some embodiments, the contrast agent comprises an iodine-based compound. In some embodiments, the iodine compound is an iodine-based small molecule. In some embodiments, the contrast agent described herein, comprises an iodine containing nanoparticle.

In some embodiments, the contrast agent is selected from T1-class and T2-class MRI contrast agents.

The phrase “MRI contrast agents” refers to a group of contrast media typically used to improve the visibility of internal body structures in magnetic resonance imaging.

The phrase “T1-class and T2-class MRI contrast agents” is used herein to denote that tissue can be characterized by two different relaxation times, typically referred to as T1 and T2. T1 (longitudinal relaxation time) is known to a skilled artisan as the time constant which determines the rate at which excited protons return to equilibrium. It is a measure of the time taken for spinning protons to realign with the external magnetic field. T2 (transverse relaxation time) is known to a skilled artisan as the time constant which determines the rate at which excited protons reach equilibrium or go out of phase with each other. It is a measure of the time taken for spinning protons to lose phase coherence among the nuclei spinning perpendicular to the main field.

In some embodiments, at least one contrast agent comprises a metal selected from a superparamagnetic metal, a diamagnetic metal, a paramagnetic metal, a ferromagnetic metal, or any combination thereof.

In some embodiments, at least one paramagnetic metal is selected from Barium (Ba), Tantalum (Ta), Tungsten (W), Dysprosium (Dy), Platinium (Pt), Gadolinium (Gd), and Manganese (Mn).

In some embodiments at least one diamagnetic metal is selected from Bismuth (Bi), and Gold (Au).

In some embodiments, the contrast agent described herein, comprises iron metal (Fe). In some embodiments, the contrast agent described herein, comprises an iron-based paramagnetic compound. In some embodiments, the iron compound is an iron oxide compound. In some embodiments, the contrast agent described herein, comprises an iron-based superparamagnetic alloy. In some embodiments, the iron-based superparamagnetic alloy is an iron-platinum alloy. In some embodiments, the ferromagnetic metal is iron (Fe).

In some embodiments, the contrast agent described herein comprises a metal ion. In some embodiments, the metal ion is selected from, without being limited thereto, gadolinium, iron, and manganese. In some embodiments, the contrast agent further comprises an organic metal coordinating compound (chelator). In some embodiments, the chelator comprises at least one metal coordinating chemical group. In some embodiments, the metal coordinating chemical group is selected from, without being limited thereto, imidazole, carboxylate, phosphate, and phosphonate. In some embodiments, the metal chelator is selected from, without being limited thereto, desferrioxamine (DFOA), tetraazacyclododecane-1,4,7,10-tetraacetic acid or gadoteric acid (DOTA), diethylenetriamine penta-acetic acid (DTPA) and dipyridoxyl diphosphate (DPDP). In some embodiments, the chelator having a high binding affinity and a specific coordination geometry towards a specific metal ion. In some embodiments, the contrast agent comprises a specific chelator-metal ion pair. In some embodiments, the specific pair is selected from desferrioxamine (DFOA)-Fe, tetraazacyclododecane-1,4,7,10-tetraacetic acid-Gd, gadoteric acid (DOTA)-Gd, diethylenetriamine penta-acetic acid (DTPA)-Mn, and dipyridoxyl diphosphate (DPDP)-Mn. In some embodiments, the chelator-metal ion complex is in the form of a cage or a metal-organic framework (MOF).

In some embodiments, the contrast agent is in the form of a MOF. In some embodiments, the contrast agent is in the form of a particle. In some embodiments, the contrast agent is in the form of an inorganic nanoparticle. In some embodiments, the inorganic nanoparticle is selected from, without being limited thereto, metallic alloys and metal oxide compounds.

In some embodiments, the hybrid molecule comprises at least one substrate of a self-labeling enzyme. In some embodiments, the enzyme is a recombinant enzyme. In some embodiments, the enzyme is a fusion protein. In some embodiments, the enzyme is a genetically-encoded enzyme that is ectopically expressed in the membranes of cells. In some embodiments, the enzyme is displayed on the external surface of the cell (i.e., exposed to the extracellular matrix). In some embodiments, the enzyme is an enzyme orthogonal to the cell or tissue. In some embodiments, the enzyme is exogenous to a target cell or tissue. In some embodiments, the enzyme is orthogonal to the substrate.

In some embodiments, the enzyme is in a fusion protein that is expressed in the target cell. In some embodiments, the enzyme is expressed in the cytoplasm of the target cell. In some embodiments, the enzyme is expressed in the ER and/or golgi of the target cell. In some embodiments, the enzyme is expressed on the plasma membrane of the target cell. In some embodiments, the enzyme is in the extracellular domain of a fusion protein that is expressed in the plasma membrane of a target cell. In some embodiments, the fusion protein comprising the enzyme further comprises at least one protein domain that anchors the enzyme in the plasma membrane. In some embodiments, the anchoring protein domain is a transmembrane domain. In some embodiments, the anchoring protein domain is a lipid anchor. In some embodiments, the lipid anchor is a glycosylphosphatidylinositol-linked protein (GPI) anchor. In some embodiments, the fusion protein comprising the enzyme comprises 1, 2, 3, 4, 5, 6, 7, 8, 9 or 10 protein domains that anchor the enzyme to the plasma membrane. In some embodiments, the enzyme is 5′ to an anchoring protein domain. In some embodiments, the enzyme is 3′ to an anchoring protein domain.

In some embodiments, the fusion protein comprising the enzyme comprises a signal peptide at the 5′ end. In some embodiments, the signal peptide is for entrance into the endoplasmic reticulum (ER). In some embodiments, the fusion protein comprising the enzyme comprises at least one protein trafficking motif. In some embodiments, the protein trafficking motif is a golgi-export and/or membrane trafficking motif. In some embodiments, the golgi-export and/or membrane trafficking motif comprises the sequence KSRITSEGEYIPLDQIDINV (SEQ ID NO: 1). In some embodiments, the protein trafficking motif is an ER export motif. In some embodiments, the ER export motif comprises the sequence FCYENEV (SEQ ID NO: 2). In some embodiments, the fusion protein comprises both an ER-export and a golgi-export motif. In some embodiments, the fusion protein comprises an ER-export motif and not a golgi-export motif. In some embodiments, the fusion protein comprises a red fluorescent moiety and comprises an ER-export motif and not a golgi-export motif. In some embodiments, the fusion protein comprises a green fluorescent moiety and comprises both an ER-export and golgi-export motif. In some embodiments, the protein trafficking motif is at the 3′ end of the fusion protein. In some embodiments, the fusion protein comprising the enzyme is optimized for surface expression of the protein.

In some embodiments, the fusion protein is configured for predominantly surface expression and the enzyme is in an extracellular portion of the protein. In some embodiments, the fusion protein is configured for predominantly expression in the ER and/or golgi and the enzyme is in an intracellular portion of the protein. In some embodiments, fusion protein is configured for cytoplasmic expression.

In some embodiments, the enzyme described herein, is designed to be expressed along with a fluorescent protein tag. Examples of fluorescent protein tags include but are not limited to green fluorescent protein (GFP), yellow fluorescent protein (YFP) and red fluorescent protein (RFP). In some embodiments, the fusion protein comprising the enzyme comprises a fluorescent moiety. In some embodiments, the fluorescent moiety is in the extracellular domain of the fusion protein. In some embodiments, the fluorescent moiety is in the intracellular domain of the fusion protein. In some embodiments, the fluorescent moiety is 5′ to the enzyme. In some embodiments, the fluorescent moiety is 3′ to the enzyme. In some embodiments, the fluorescent moiety is 5′ to the anchoring protein domain. In some embodiments, the fluorescent moiety is 3′ to the anchoring protein domain. In some embodiments, the fluorescent moiety is 5′ to the ER export motif. In some embodiments, the fluorescent moiety is 3′ to the ER export motif. In some embodiments, the fluorescent moiety is a different fluorescent moiety than the fluorescent moiety of the hybrid molecule. In some embodiments, the fluorescent moiety of the fusion molecule and the hybrid molecule can be separately imaged.

In some embodiments, the self-labeling enzyme is capable of covalently binding to the substrate. In some embodiments, the self-labeling enzyme is selected from, without being limited thereto, a SNAP-tag, a CLIP-tag, and a Halo-tag. In some embodiments, the self-labeling enzyme irreversibly binds to its substrate. In some embodiments, the self-labeling enzyme reversibly binds to its substrate.

The term “SNAP-tag” refers to a mutant of the DNA repair protein 0⁶-alkylguanine-DNA alkyltransferase that reacts specifically and rapidly with benzylguanine (BG) derivatives.

The term “CLIP-tag” refers to a protein that was created by engineering the substrate specificity of the SNAP-tag, permitting it to react specifically with 0²-benzylcytosine (BC) derivatives.

The term “Halo-tag” refers to a bacterial hydrolase enzyme, which has a genetically modified active site, which specifically binds the reactive chloroalkane substrate. The reaction that forms the bond between the protein tag and chloroalkane substrate is fast and essentially irreversible under physiological conditions due to the terminal chlorine of the substrate.

In some embodiments, the substrate described herein, is selected from, without being limited thereto, O⁶-benzylguanine derivatives, O²-benzylcytosine derivatives, and chloroalkane derivatives. In some embodiments, the enzyme/substrate pair is selected from SNAP-tag/O⁶-benzylguanine, CLIP-tag/O²-benzylcytosine, and Halo-tag/chloroalkane derivatives.

In some embodiments, the hybrid molecule further comprises at least one fluorescent moiety. In some embodiments, the fluorescent moiety comprises a fluorescent dye or a fluorophore. In some embodiments, the fluorescent moiety is capable of emitting ultra-violet (UV) light. In some embodiments, the fluorescent moiety is capable of emitting near infrared (IR) light. In some embodiments, the fluorescent moiety is capable of emitting infrared light. In some embodiments, the fluorescent moiety is capable of emitting visible light. In some embodiments, the fluorescent moiety is capable of emitting UV, IR, near-IR and/or visible light. In some embodiments, the fluorescent moiety is selected from, without being limited thereto, fluorescein, diacetylfluorescein, dipivaloyl Oregon green, red fluorescent probe Cy3, tetramethylrhodamine, far-red fluorescent Cy5, Alexa Fluor-dyes, Atto-dyes, BODIPY-dyes, Rhodamine-dyes (and Rhodamine silicone derivatives), GFP, enhanced GFP (eGFP), YFP, RFP, mCherry, Tomato and a quantum dot. In some embodiments, the fluorescent moiety is selected from GFP and Tomato.

In some embodiments, the fluorescent moiety is at the 5′ end of the hybrid molecule. In some embodiments, the fluorescent moiety is between the contrast agent and the substrate. In some embodiments, the fluorescent moiety is at the 3′ end of the hybrid molecule. In some embodiments, the fluorescent moiety is separated from the contrast agent, the substrate or both by a linker. In some embodiments, the linker is a protein linker. In some embodiments, the linker is a hydrophobic linker. In some embodiments, the linker is used to increase permeability of the hybrid molecule.

In some embodiments, the fluorescent moiety is hydrophobic. In some embodiments, the fluorescent moiety is used to increase hydrophobicity of the hybrid molecule. In some embodiments, the hybrid molecule comprises more than one copy of the fluorescent moiety in order to increase hydrophobicity and/or permeability.

In some embodiments, the hybrid molecule, has the general Formula:

(R)ₙ—(F)ₘ—(S)ₚ

wherein: R represents the contrast agent, described herein throughout. In some embodiments, F represents the fluorescent moiety, described herein throughout. In some embodiments, S represents the substrate, described herein throughout. In some embodiments, n, and m, and p are each, independently, an integer from 1 to 5. In some embodiments, n and p are each, independently, an integer from 1 to 5 and m is an integer from 0 to 5. In some embodiments, n:m:p value is 1:0:1, 1:1:1, 2:0:1, 2:1:1, 3:0:1, 3:1:1, 4:0:1, 4:1:1, 1:2:1, 1:3:1, 1:4:1, 2:2:1, 3:3:1, 4:4:1, 3:4:1, 4:3:1, 2:3:1, 3:2:1 or any value therebetween. In some embodiments, n:m:p value is 1:1:1. In some embodiments, n:m:p value is 1:0:1.

In some embodiments, the hybrid molecule comprises 1, 2, 3, 4, or 5 contrast agents. Each possibility represents a separate embodiment of the invention. In some embodiments, the multiple contrast agents are all the same agent. In some embodiments, the hybrid protein comprises multiple copies of the same contrast agent. In some embodiments, the hybrid molecule does not comprise a plurality of different contrast agents. In some embodiments, the hybrid molecule comprises a plurality of different contrast agents.

In some embodiments, the hybrid molecule comprises 1, 2, 3, 4, or 5 fluorescent moieties. Each possibility represents a separate embodiment of the invention. In some embodiments, the multiple fluorescent moieties are all the same moiety. In some embodiments, hybrid molecule does not comprise a plurality of different fluorescent moieties. In some embodiments, the hybrid molecule comprises a plurality of different fluorescent moieties.

In some embodiments, the hybrid molecule comprises 1, 2, 3, 4, or 5 substrates. Each possibility represents a separate embodiment of the invention. In some embodiments, the multiple substrates are all the same molecule. In some embodiments, the hybrid molecule does not comprise a plurality of different substrates. In some embodiments, the hybrid molecule comprises a plurality of different substrates.

In some embodiments, R, F, and S in the formula described are covalently linked via a molecular linker. One skilled in the art will appreciate that the linker may be selected from various molecular linkers that do not sterically hinder the activity of the various moieties. In some embodiments, the linker is a hydrocarbon. In some embodiments, the linker is a linear hydrocarbon. In some embodiments, the hydrocarbon consists of 1, 2, 3, 4, 5, 6, or 7 carbon atoms. In some embodiments, the molecular linker is a polymer including but not limited to polyethylene glycol.

In some embodiments, the hybrid molecule comprises or consists of molecule 1 as described herein below. In some embodiments, the hybrid molecule comprises or consists of molecule 2 as described herein below. In some embodiments, the hybrid molecule lacks a fluorescent moiety and comprises or consists of molecule 1. In some embodiments, the hybrid molecule comprises a fluorescent moiety and comprises or consists of molecule 2.

In some embodiments, the hybrid molecule is for use in a cell-specific imaging assay. In some embodiments, the imaging assay is selected from magnetic resonance imaging (MRI), X-ray, computed tomography (CT)-scan, and microscopy. In some embodiments, the microscopy is selected from electron microscopy (EM), fluorescent microscopy, and atomic force microscopy. In some embodiments, the hybrid molecule is for use in imaging a specific target cell. In some embodiments, the cell is a human cell. In some embodiments, the cell is selected from, without being limited thereto, a brain cell, a liver cell, a heart cell, a lung cell, a blood cell, and a bone marrow cell. In some embodiments, the cell is a neuronal cell. In some embodiments, the cell is a cancer cell. In some embodiments, the cell is an immune cell.

In some embodiments, the hybrid molecule may be used as a theranostics tool. In some embodiments, the hybrid molecule may be used for theranostics. In some embodiments, the hybrid molecule comprises a therapeutic agent. In some embodiments, the contrast agent is a therapeutic agent. In some embodiments, the therapeutic agent is gold. In some embodiments, the therapeutic agent is a radioactive agent. In some embodiments, the radioactive agent is a radiolabel. In some embodiments, there therapeutic agent is inserted via a linker into the hybrid molecule. In some embodiments, the therapeutic agent is inserted before the contrast agent, before the fluorescent moiety, before the substrate, at the 5′ end, or at the 3′ end. Indeed, a skilled artisan will appreciate that the therapeutic agent can be inserted at any location in the hybrid molecule.

According to another aspect, the present invention provides a composition comprising a hybrid molecule of the invention and a carrier, excipient or adjuvant. In some embodiments, the composition is a pharmaceutical composition. In some embodiments, the composition is a diagnostic composition. In some embodiments, the composition is formulated for administration to a subject. In some embodiments, the subject is a mammal. In some embodiments, the subject is a human.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the compositions presented herein.

In some embodiments, the composition comprises at least a first and a second hybrid molecule. In some embodiments, the fluorescent moiety and substrate of the first hybrid molecule differs from the fluorescent moiety and substrate of the second hybrid molecule. In some embodiments, the substrate of the first hybrid molecule differs from the substrate of the second hybrid molecule. In some embodiments, the substrate of the first and second hybrid molecules are the same. In some embodiments, the fluorescent moiety of the first molecule is different than the fluorescent moiety of the second molecule. In some embodiments, the fluorescent moiety of the first and second molecules is the same. In some embodiments, the contrast agent of the first molecule is different than the contrast agent of the second molecule. In some embodiments, the contrast agent of the first and second molecules is the same. In some embodiments, at least one of the fluorescent moiety and the contrast agent of the first hybrid molecule is different than the fluorescent moiety and contrast agent of the second hybrid molecule. In some embodiments, first and second hybrid molecules comprises a similar magnetic contrast agent. In some embodiments, the magnetic contrast agent of the first hybrid molecule differs from the magnetic contrast agent of the second hybrid molecules.

According to another embodiment, the present invention provides a cell-specific imaging method. According to another embodiment, the present invention provides a dual paramagnetic-fluorescent imaging method. According to another embodiment, the present invention provides a method of imaging a target cell or a specific target cell.

In some embodiments, the method comprises contacting one or more cells with a composition comprising a hybrid molecule of the invention. In some embodiments, at least one cell or one specific cell of the one or more cells expresses of one or more self-labeling enzymes. In some embodiments, at least one cell or one specific cell of the one or more cells comprise membranal expression of one or more self-labeling enzymes. In some embodiments, at least one cell, or one specific cell, of the one or more cells express on its surface one or more self-labeling enzymes. In some embodiments, the enzyme is orthogonal to the hybrid molecule. In some embodiments, the enzyme binds the substrate that is part of the hybrid molecule. In some embodiments, the enzyme is orthogonal to the substrate. In some embodiments, the enzyme binds the substrate. In some embodiments, at least one cell or one specific cell of the one or more cells expresses a fusion protein as described herein. In some embodiments, the cell expressing the fusion protein, or the enzyme, is the target cell.

In further embodiment, a magnetic field is applied to the one or more cells. In further embodiments, a light is applied to the one or more cells. In some embodiments, the light is configured to cause a fluorescent moiety to fluoresce. In some embodiments, the light is configured to cause the fluorescent moiety of the hybrid molecule to fluoresce. In some embodiments, the light is configured to cause the fluorescent moiety of the fusion protein to fluoresce. In some embodiments, more than one light is applied, and the lights are configured to cause the fluorescent moieties of the hybrid molecule and fusion protein to fluoresce. In some embodiments, the light applied is from a microscope. In some the magnetic field is from an MRI. In some embodiments, the magnetic field is from a CT-scan. In some embodiments, X-rays are applied to the one or more cells.

In some embodiments, the one or more cells described herein throughout are in the form of a tissue. In some embodiments, the one or more cells are in a subject. In some embodiments, the one or more cells are in a subject in need of imaging of a target cell. In some embodiments, the one or more cells are in a subject in need of a diagnosis concerning a target cell. In some embodiments, the one or more cells are in a difficult to image area of a subject. In some embodiments, the one or more cells are in an area of a subject to which a standard contrast dye does not reach. In some embodiments, the cells in culture. In some embodiments, the cells are ex-vivo.

In some embodiments, the contacting described herein is performed ex-vivo. In some embodiments, the contacting described herein is performed in-vivo. In some embodiments, in vivo examination of a specific cell type is applied, at any tissue depth, non-invasively and by non-damaging means. In some embodiments, in vivo examination of a labeled cancer cell, being tracked throughout the body. In some embodiments, the cancer cell, is further analyzed for tissue infiltration.

In some embodiments, the method further comprises detecting or imaging the hybrid molecule. In some embodiments, the contrast agent is detected or imaged. In some embodiments, a fluorescent moiety is detected or imaged. In some embodiments, both a contrast agent and a fluorescent moiety is detected or imaged. In some embodiments, the contrast agent and fluorescent moiety are imaged at the same time or one before the other. In some embodiments, the fusion protein in the target cell is also detected or imaged. In some embodiments, the fluorescent moiety in the fusion protein in the target cell is detected or imaged.

In some embodiments, the method comprises a preliminary step of expressing within one or more cells, the self-labeling enzymes. In some embodiments, the method further comprises expressing within a target or specific target cell a fusion protein as described herein. As one skilled in the art will appreciate, said expression step may take place in-vivo or ex-vivo. In some embodiments, a target cell is made to express the fusion protein and then administered to the subject. In some embodiments, a cell is extracted from the subject, made to express the fusion protein and then administered to the subject. In some embodiments, a sufficient amount of time is allowed to pass such that the administered cell reaches a target area in the subject. In some embodiments, after administering the cell expressing the fusion protein the cell is allowed to migrate to a target area to be imaged.

In some embodiments, the administered cell is allogenic to the subject. In some embodiments, the cell is autologous to the subject. In some embodiments, the administered cell is non-immunogenic. It will be understood by one skilled in the art that there are cells that do not express MHC class II molecule and thus can be from a different individual but not elicit an immune response when transferred to the subject. In some embodiments, the administered cell is selected from an immune cell, a cancer cell, a stem cell, a mesenchymal stem cell, a bone marrow cell, and an umbilical cord cell. In some embodiments, the administered cell is an immune cell. In some the administered cell is a cancer cell taken from the subject. In some embodiments, the administered cell is a stem cell. In some embodiments, the administered cell is a cell that homes to a location in the subject. It will be understood by a skilled artisan, that a number of cells home to areas in the body, such as areas of inflammation, tumors, specific organs and the like. Such a cell may be used to image such a location.

In some embodiments, the self-labeling enzyme is orthogonal to the cells being targeted. In some embodiments, the self-labeling enzyme is ectopically expressed in the cells being targeted. In some embodiments, the self-labeling enzyme is part of a synthetical protein, or fusion protein that is exogenously expressed in the cells being targeted.

In some embodiments, the expression described herein, comprises or consists of introducing into the target cell a nucleic acid construct comprising a polynucleotide sequence encoding a self-labeling enzyme. In some embodiments, expressing comprises introducing into the target cell a nucleic acid molecule that encodes a fusion protein described herein. In some embodiments, the nucleic acid molecule is a vector that comprises a polynucleotide sequence that encodes a fusion protein described herein. In some embodiments, the vector is an expression vector.

In some embodiments, the polynucleotide sequence encoding a self-labeling enzyme is operatively linked to a promoter. In some embodiments, the promoter is a cell type-specific promoter. In some embodiments, the cell-specific promoter is a tissue-specific promoter. In some embodiments, the promoter is a neuron specific promoter. In some embodiments, a generic promoter is used and it nucleic acid molecule comprises a tissue specific regulatory element. In some embodiments, the regulatory element is an enhancer. In some embodiments, the regulatory element is a miR binding site. In some embodiments, the nucleic acid molecule comprises an element that restrict expression of the fusion protein to a specific target cell.

Tissue- and cell type-specific promoters are well known in the art, as are regulatory elements that restrict expression to specific cells/tissues. The invention may be performed with any such elements, that will target production of the fusion protein to only the area/tissue/cells that are to be imaged. Such specific promoters may be used when the nucleic acid molecule will be administered systemically and thus requires restricted expression. If ex-vivo cells are given the nucleic acid molecule, then restricted expression may not be needed, though it may still be employed.

In some embodiments, the introducing step described herein, may be applied via a transfection, an infection or a transformation. In some embodiments, the transfection described herein, comprises using a transfecting vector. In some embodiments, the vector is a cell-specific. In some embodiments, the vector is a virus. In some embodiments, the nucleic acid molecule is introduced into a viral vector and viral particles are produced for infection.

In some embodiments, the introducing is performed systemically to a subject. In some embodiments, the systemic introducing is by viral infection. In some embodiments, the virus infects only target cells. In some embodiments, the virus infects all cells. In some embodiments, the virus infects all cells, but the fusion protein is only expressed in target cells. This can be achieved by a variety of methods such as cell type-specific promoters or other methods such as are described herein.

In some embodiments, the nucleic acid construct described herein, further comprises a polynucleotide sequence encoding one or more fluorescent proteins. In some embodiments, the two proteins described herein (i.e. self-labeling enzyme and fluorescent protein) are genetically encoded under a single promoter. In some embodiments, the two proteins described herein are expressed as a fusion protein. In some embodiments, the two proteins described herein are genetically encoded under different promoters. In some embodiments, the fluorescent protein provides an optical signal for transfected cells. In some embodiments, the fluorescent protein provides an optical signal for self-labeling enzyme expressing cells. In some embodiments, the fluorescent protein is selected from a green fluorescent protein and a red fluorescent protein.

In some embodiments, the nucleic acid comprises one, two, three, four or five repeats of a single type of a self-labeling enzyme (e.g., SNAP, CLIP or HaLo) thereby creating a polymer able to bind multiple molecules at once. One skilled in the art will appreciate that different polypeptides can be expressed in the target tissue to provide strong contrast and resolution for two distinct signals arising from different hybrid molecules of the invention.

“Nucleic acid” refers to a molecule which can be single stranded or double stranded, composed of monomers (nucleotides) containing a sugar, phosphate and either a purine or pyrimidine. In bacteria, lower eukaryotes, and in higher animals and plants, “deoxyribonucleic acid” (DNA) refers to the genetic material while “ribonucleic acid” (RNA) is involved in the translation of the information from DNA into proteins.

The terms “polypeptide,” “peptide” and “protein” as used herein are used interchangeably to refer to a polymer of amino acid residues. The term also applies to amino acid polymers in which one or more amino acids are chemical analogues or modified derivatives of a corresponding naturally-occurring amino acids.

The term “expressed” as used herein is intended to mean the transcription and translation to gene product from a gene coding for the sequence of the gene product.

The phrase “ectopically expressed” refers to an abnormal gene expression in a cell type, tissue type, or developmental stage in which the gene is not usually expressed, or the protein of the gene is not functional.

As used herein, a “vector”, “expression vector” or “plasmid” as referred to herein is an extra-chromosomal element often carrying genes which are not part of the central metabolism of the cell, and usually in the form of circular double-stranded DNA molecules. It may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. An expression vector is one into which a desired DNA sequence may be inserted by restriction and ligation such that it is operatively joined to regulatory sequences and may be expressed as an RNA transcript. Vectors may further contain one or more marker sequences suitable for use in the identification and selection of cells which have been transformed or transfected with the vector. As used herein, “transformation” or “transfection” is the acquisition of new genes in a cell by the incorporation of nucleic acid. Markers include, for example, genes encoding proteins which increase or decrease either resistance or sensitivity to antibiotics or other compounds, genes which encode enzymes whose activities are detectable by standard assays known in the art (e.g., 0-galactosidase or alkaline phosphatase), and genes which visibly affect the phenotype of transformed or transfected cells, hosts, colonies or plaques.

As indicated above, the expression vector of the invention may be operatively linked to a promoter. The terms “promoter” refer to a sequence of DNA, usually upstream of (5′ to) the protein coding sequence of a structural gene, which controls the expression of the coding region by providing the recognition for RNA polymerase and/or other factors required for transcription to start at the correct site. Promoter sequences are necessary but not always sufficient to drive the expression of the gene.

A coding sequence and regulatory sequences are the to be “operatively linked” when they are covalently linked in such a way as to place the expression or transcription of the coding sequence under the influence or control of the regulatory sequences. If the regulatory sequence is positioned relative to the gene such that the regulatory sequence is able to exert a measurable effect on the amount of gene product produced, then the regulatory sequence is operatively linked to the gene. If it is desired that the coding sequences be translated into a functional protein, two DNA sequences are the to be operatively joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operatively joined to a coding sequence if the promoter region were capable of effecting transcription of that DNA sequence such that the resulting transcript might be translated into the desired protein or polypeptide.

In some embodiments, ex-vivo contacting enables avoiding the injection of a virus to a subject, e.g., a human. In some embodiments, a sample comprising cells is contacted with the nucleic acid construct. In some embodiments, the cells are then introduced to the subject. In some embodiments, the cells are then imaged by MRI. In some embodiments, the cells may be assessed for their ability to infiltrate different tissues. In some embodiments, the cells may be assessed for their ability to infiltrate the brain.

In one embodiment, proteins of the present invention are inserted into expression vectors (i.e., a nucleic acid construct) to enable expression of the recombinant protein. In one embodiment, the expression vector of the present invention includes additional sequences which render this vector suitable for replication and integration in prokaryotes. In one embodiment, the expression vector of the present invention includes additional sequences which render this vector suitable for replication and integration in eukaryotes. In one embodiment, the expression vector of the present invention includes a shuttle vector which renders this vector suitable for replication and integration in both prokaryotes and eukaryotes. In some embodiments, cloning vectors comprise transcription and translation initiation sequences (e.g., promoters, enhances) and transcription and translation terminators (e.g., polyadenylation signals).

In one embodiment, the expression vector of the present invention can further include additional polynucleotide sequences that allow, for example, the translation of several proteins from a single mRNA such as an internal ribosome entry site (IRES) and sequences for genomic integration of the promoter-chimeric protein.

In some embodiments, mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMT010/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors are useful for in vivo expression of the proteins of the present invention since they offer advantages such as lateral infection and targeting specificity. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In one embodiment, various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, introduction of nucleic acid by viral infection offers several advantages over other methods such as lipofection and electroporation, since higher transfection efficiency can be obtained due to the infectious nature of viruses. In some embodiments, viral infection of a subject is used to introduce the fusion protein.

In one embodiment, it will be appreciated that the proteins of the present invention can also be expressed from a nucleic acid construct administered to the individual employing any suitable mode of administration. In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

It will be appreciated that other than containing the necessary elements for the transcription and translation of the inserted coding sequence (encoding the protein), the expression construct of the present invention can also include sequences engineered to optimize stability, production, purification, yield or activity of the expressed protein.

Various methods, in some embodiments, can be used to introduce the expression vector of the present invention into the host cell system. In some embodiments, such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

The Kit

According to some embodiments of the present invention there is provided a kit, comprising (a) at least one hybrid molecule, described herein throughout or the composition comprising a hybrid molecule, described herein throughout, and (b) at least one nucleic acid molecule comprising a polynucleotide sequence encoding one or more self-labeling enzymes. In some embodiments the nucleic acid molecule is a vector such as is described herein.

In some embodiments, the kit comprises at least one hybrid molecule, described herein throughout at least one nucleic acid construct comprising a polynucleotide sequence encoding one or more self-labeling enzyme. In some embodiments, the kit comprises at least one hybrid molecule, described herein throughout at least one nucleic acid construct comprising a polynucleotide sequence encoding one or more self-labeling enzyme linked to a cell-specific promoter, described herein throughout.

In some embodiments, the kit comprises two different hybrid molecules, described herein throughout. In some embodiments, the kit comprises a composition comprising two different hybrid molecules, described herein throughout.

In some embodiments, the kit comprises at least 1, 2, 3, 4, or 5 hybrid molecules. Each possibility represents a separate embodiment of the invention. In some embodiments, the kit comprises a plurality of hybrid molecules. In some embodiments, the kit comprises at least two different contrast agents. In some embodiments, the kit comprises at least two different fluorescent moieties. In some embodiments, the kit comprises at least two different substrates. In some embodiments, the kit comprises at least a first molecule comprising a first substrate and a second molecule comprising a second substrate, and wherein the first and second substrates are different. In some embodiments, the kit comprises at least a first molecule comprising a first contrast agent and a second molecule comprising a second contrast agent, and wherein the first and second contrast agents are different. In some embodiments, the kit comprises at least a first molecule comprising a first fluorescent moiety and a second molecule comprising a second fluorescent moiety, and wherein the first and second fluorescent moieties are different.

According to some embodiments, the kit is utilized by contacting at least one hybrid molecule, described herein throughout and one nucleic acid construct comprising a polynucleotide sequence encoding one or more self-labeling enzyme with one or more cells and applying a magnetic field, X-radiation, UV-vis light or any combination thereof, on the cells. In some embodiments, the kit is for use in an imaging assay. In some embodiments, the kit is for use in methods such as are described herein.

In some embodiments, the kit comprises instructions for use. In some embodiments, the kit comprises instructions for further designing the nucleic acid molecule to optimize expression in a target cell, in a target location in a target cell or both.

In some embodiments of the subject kits, the composition of two different hybrid molecules, described herein throughout, and the nucleic acid construct comprising a polynucleotide sequence encoding one or more self-labeling enzyme optionally operatively linked to a cell-specific promoter, described herein throughout are packaged within a container.

In some embodiments, the container is made of a material selected from the group consisting of thin-walled film or plastic (transparent or opaque), paperboard-based, foil, rigid plastic, metal (e.g., aluminum), glass, etc.

In some embodiments, the content of the kit is packaged, as described below, to allow for storage of the components until they are needed.

In some embodiments, some or all components of the kit may be packaged in suitable packaging to maintain sterility.

In some embodiments of the subject kits, the hybrid molecules, described herein throughout, and the nucleic acid construct comprising a polynucleotide sequence encoding one or more self-labeling enzyme are stored in separate containers within the main kit containment element e.g., box or analogous structure, may or may not be an airtight container, e.g., to further preserve the sterility of some or all of the components of the kit.

In some embodiments, the dosage amount of the one or more hybrid molecules and one or more nucleic acid construct comprising a polynucleotide sequence encoding one or more self-labeling enzyme provided in a kit may be sufficient for a single application or for multiple applications.

In those embodiments, the kit may have multiple dosage amounts of the one or more hybrid molecules and one or more nucleic acid construct comprising a polynucleotide sequence encoding one or more self-labeling enzyme packaged in a single container, e.g., a single tube, bottle, vial, Eppendorf and the like.

In some embodiments, the kit may have multiple dosage amounts of one or more hybrid molecules and one or more nucleic acid construct comprising a polynucleotide sequence encoding one or more self-labeling enzyme individually packaged such that certain kits may have more than one container of one or more bacteria and one or more fermenting microorganism.

In some embodiments, multiple dosage amounts of the one or more hybrid molecules and one or more nucleic acid construct comprising a polynucleotide sequence encoding one or more self-labeling enzyme may be packed in single separate containers.

In some embodiments, the kit contains instructions for preparing the composition used therein and for how to practice the methods of the invention.

In some embodiments, the kit further comprises a measuring utensil such as measuring spoon or a measuring cup.

In some embodiments, the instructions may be recorded on a suitable recording medium or substrate. For example, the instructions may be printed on a substrate, such as paper or plastic, etc.

In some embodiments, the instructions may be present in the kit as a package insert, in the labeling of the container of the kit or components thereof (i.e., associated with the packaging or sub-packaging) etc. In other embodiments, the instructions are present as an electronic storage data file present on a suitable computer readable storage medium. In other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

General

As used herein the term “about” refers to ±10%.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. Any particular embodiment of the invention may include a plurality of “optional” features unless such features conflict.

As used herein and in the appended claims, the singular forms “a,” “and,” and “the” include plural referents unless the context clearly dictates otherwise.

Also, the use of “or” means “and/or” unless stated otherwise. Similarly, “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of”

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1: Designing a Novel Hybrid Paramagnetic-Fluorescent Molecule

A novel magneto-fluorescent contrast agent (MFS) having three distinct functional chemical headgroups is provided.

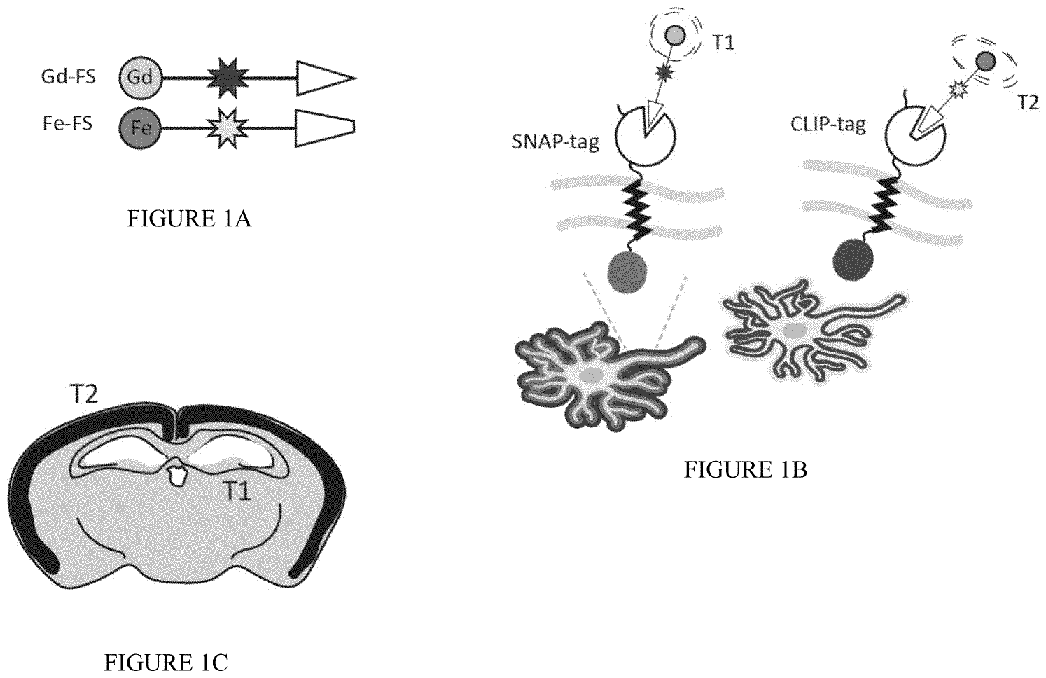

One end of the molecule consists of a strong MRI-compatible contrast agent (FIG. 1A). Optionally, each hybrid molecule has a different class of MR agents (e.g., a first T1 contrast agent, providing a positive, bright signal, and a second T2 agent that causes a negative, dark signal). Several agents are commonly used, namely gadolinium and iron, which are positive and negative magnetic (M) contrast agents, respectively. Each M-agent is incorporated into a in separate molecule (FIG. 1A, Gd—FS and Fe—FS).

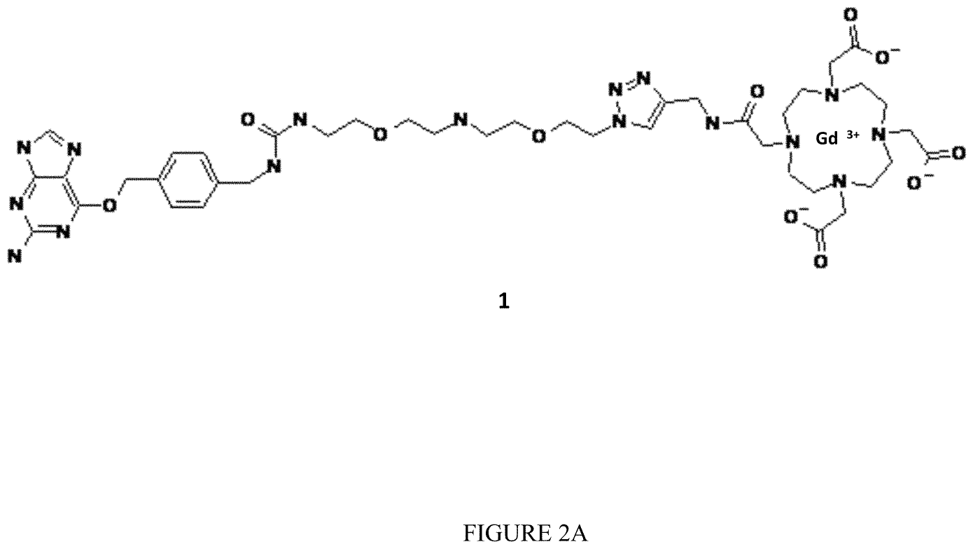

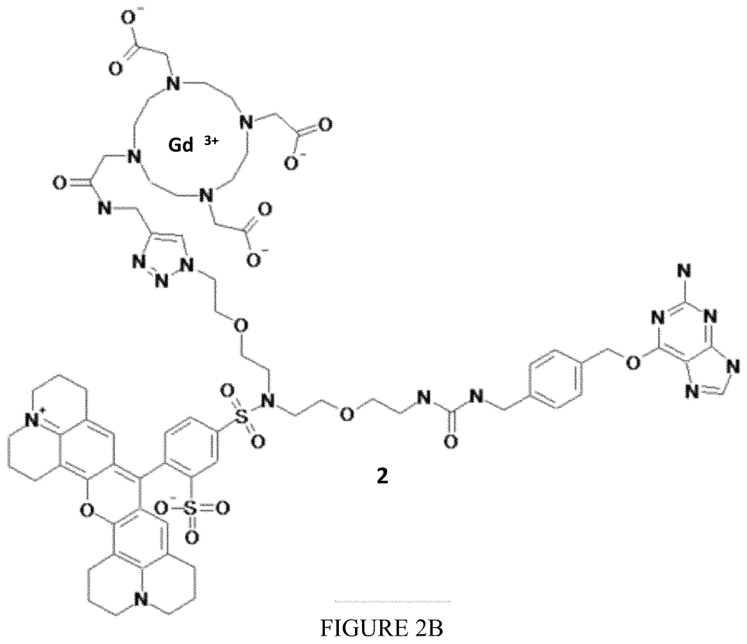

The core of the molecule consists of a synthetic fluorescent (F) molecule, whereas the other end of the molecule includes a unique enzyme substrates (S), to specifically tether the MFS to genetically-defined neuronal populations (FIG. 1A). The fluorescent molecule is not essential, and a molecule can be made with just the contrast agent and the unique enzyme substrate (See molecule 1 in FIG. 2A).

### Example 2: Engineering Cell-Specific, Substrate Immobilizing Proteins

To immobilize MFS-agents onto the surface of targer cells (e.g., neurons), self-labeling enzymes such as SNAP, CLIP and HaLo tags are used. These are small, globular enzymes, engineered to specifically, and irreversibly, react with their unique substrates; thereby acting as self-labeling suicide enzymes. When the enzymes are specifically expressed in defined cell populations and exposed to their substrates, they subsequently ‘tag’ the cells with the substrate. If this substrate is further decorated with functional headgroups, such as fluorescent dyes, the select cells will be fluorescently-labeled (FIG. 1B).

The nucleic acid molecule that encodes these tags may be composed of a single polypeptide that includes up to five repeats of a single type of a self-labeling enzyme (SNAP, CLIP or HaLo), or may comprise a promotor configured to provide high expression levels of the self-labeling enzymes, thereby creating a polymer able to bind to multiple molecules at once. Different polypeptides can be expressed in the tissue to provide strong contrast and resolution for two distinct signals arising from different hybrid molecules of the invention.

This methodology, along with the developed unique MFSs, enables imaging defined cell populations (e.g., specific neurons of the brain) by MRI and fluorescence microscopy. Importantly, the expression of two distinct enzymes in different populations of neurons, specifically tagged by two different MFSs, allows a skilled technician to distinguish between the two neuronal populations by MRI (by T1 and T2 sequences, FIG. 1C), concomitantly.

### Example 3: Characterization of the MFS-Agents

Two MFS-agents were characterized.

Gadolinium (3+) ion molecule 1: 2-[4-(1[(1-{2-[2-({2-[2-({[(4-{[(2-amino-9H-purin-6yl)oxy]methyl phenyl)methyl]carbamoyl}amino)ethoxy]ethyl}amino)ethoxy]ethyl}-1H-,2,3-triazol-4-yl)methyl]carbamoyl}methyl)-7,10-bis(carb oxymethyl)-1,4,7,10-etraazacyclododecan-1-yl]acetate, 95% (Formula: C₄₁H₅₉ GdN₁₆O₁₁), molecular weight: 1109.2578, State: crystalline powder, color: yellow (FIG. 2A).

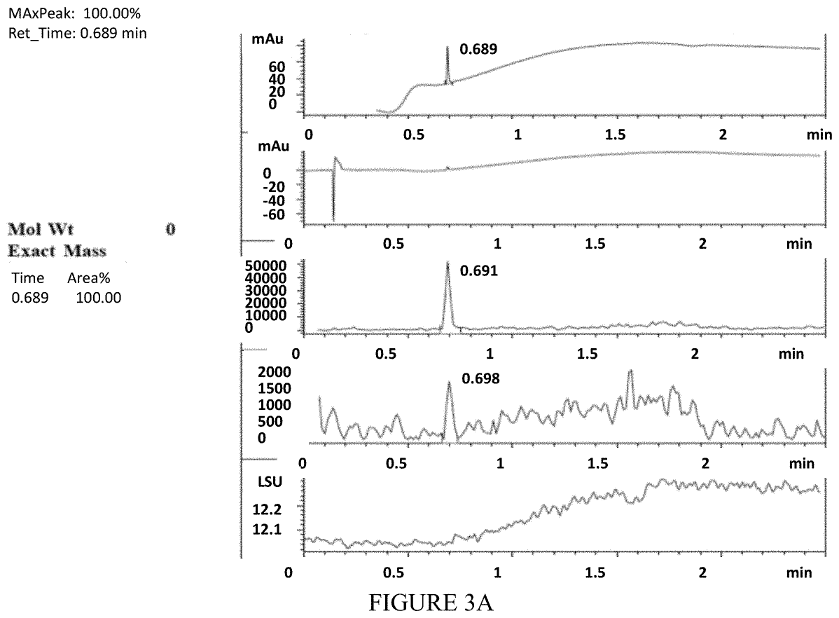

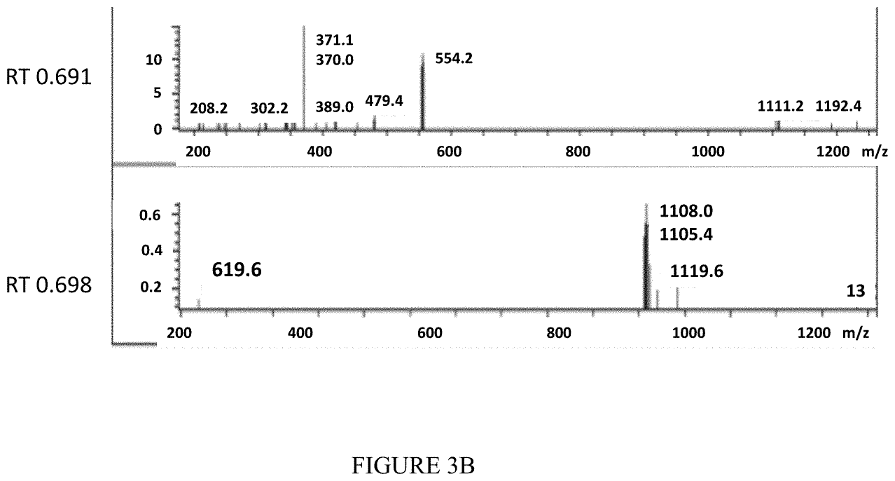

Validation of the structure was done with liquid chromatography-mass spectrometry (LCMS) which confirmed the structure (FIG. 3A-B).

Gadolinium (3+) ion molecule 2: 16-[4-({2-[2-({[(4-{[(2-amino-9H-purin-6-yl)oxy]methyl}phenyl)methyl]carbamoyl}amino)ethoxy]ethyl}(2-{2-[4-({2-[4,7,10-ris(carboxymethyl)-1,4,7,10-tetraazacyclododecan-1-yl]acetamido}methyl)-1H-1,2,3-triazol-yl]ethoxy}ethyl)sulfamoyl)-2-sulfophenyl]-3-oxa-9lambda5,23-diazaheptacyclo[17.7.1.1\hat{ }{5,9}.0\hat{ }{2,17}.0\hat{ }{4,15}.0\hat{ }{23,27}0.0{13,28}]octacosa-1,4,9(28),13,15,17,19(27)-heptaen-9-ylium, 95%. Molecular weight-1697.95. State: crystalline powder, color: dark purple. (FIG. 2B).

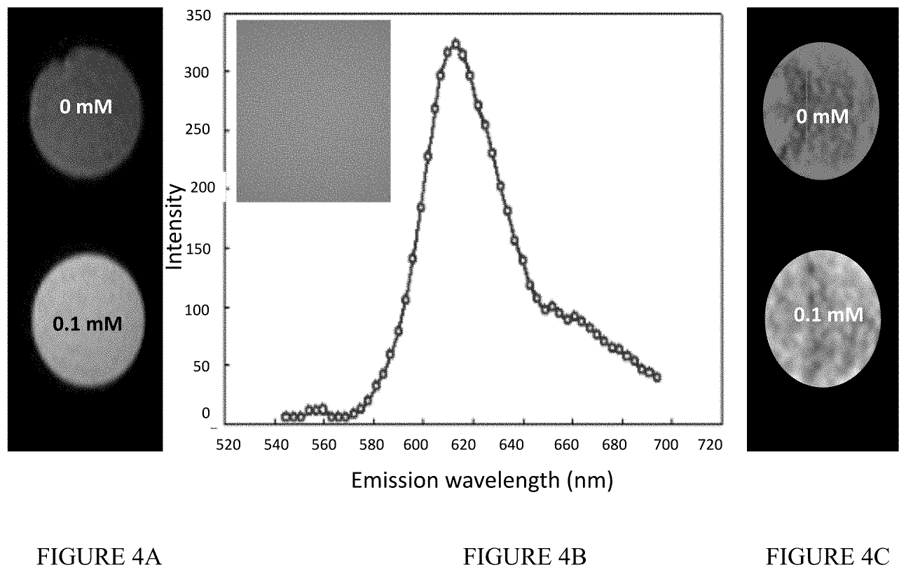

To examine the multi-functionality of the complete MFS-agent 2 (FIG. 2B), compound 2 was solubilized in water (0.1 mM) and imaged via three distinct imaging modalities (FIG. 4A-C). Even with the high hydrophobicity of the fluorescent moiety the molecule dissolved easily in water with no need to add DMSO or other solvents. This is a highly desirable trait, for a molecule that will be delivered through the blood stream. The sample was imaged using T1 MRI and very bright contrast was obtained (FIG. 4A). Red fluorescence emission was determined by using spectral analysis of a confocal microscope (FIG. 4B). The sample was also imaged with a micro-CT, in order to obtain a very strong CT signal (strong X-ray attenuation, FIG. 4C).

### Example 4: eTags

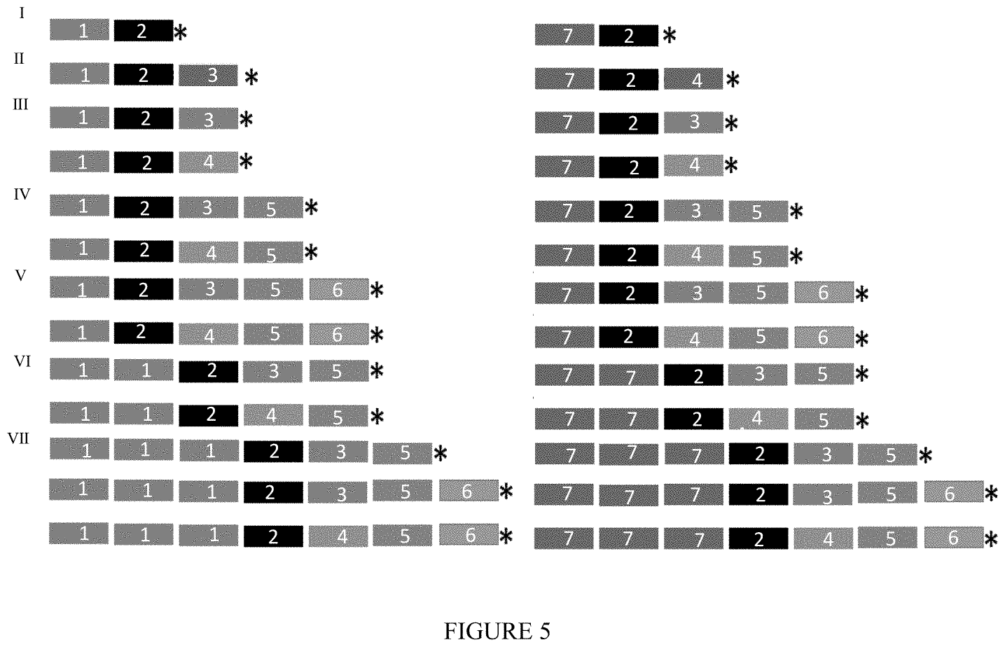

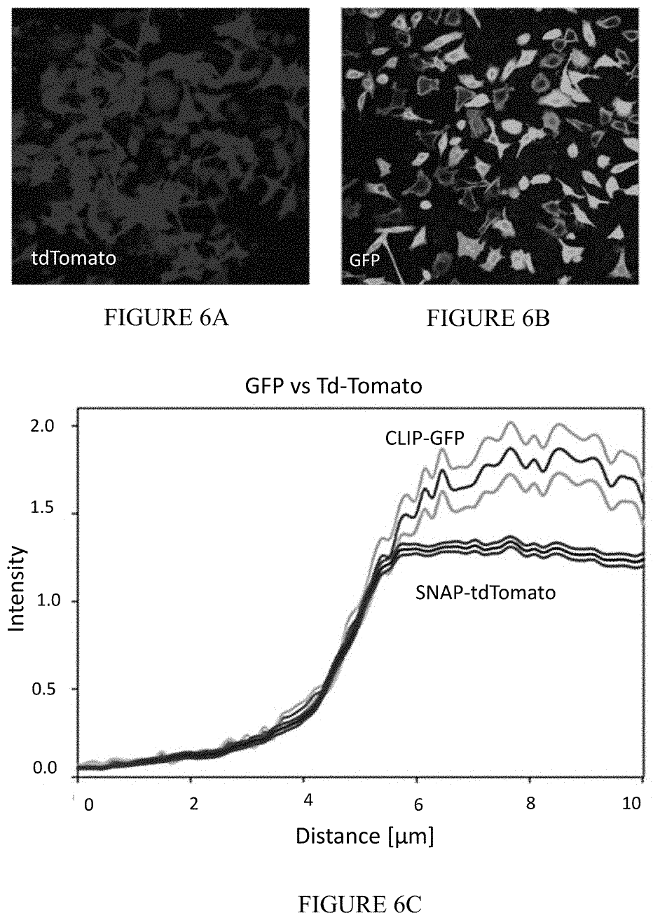

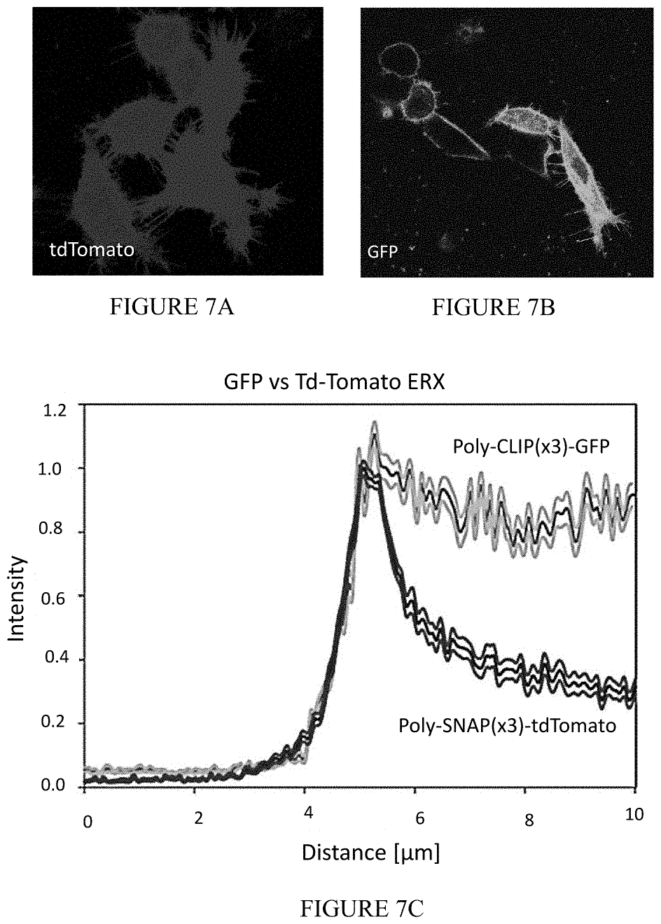

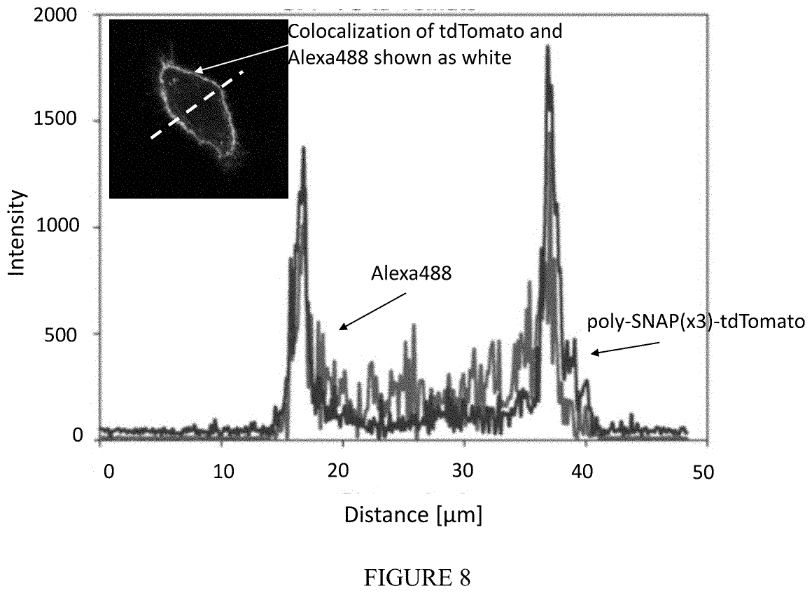

A palette of eTag and poly-eTag fusion proteins was created (FIG. 5). This was achieved by inserting either SNAP (represented by squares with number 1) or CLIP (represented by squares with number 7) domains after a signal peptide (to insert protein into membrane). These were followed by a linker and an antibody epitope (myc-tag), immediately followed by a transmembrane (TM) domain (represented by squares with number 2). Intracellularly these constructs contain either a Green- or Red-Fluorescent Proteins (GFP or tdTomato, respectively). Despite the presence of a signal peptide and TM domain, initial constructs (Group III of FIG. 5) remained predominantly intracellular (FIG. 6A-C). Close examination of the protein expression appeared to show that the fusion proteins were trapped in the ER and golgi. After several rounds of optimization, it was determined that the addition of membrane trafficking and ER-exit motifs greatly increased surface expression. Two primary motifs were used, the golgi-export and membrane trafficking signal KSRITSEGEYIPLDQIDINV (SEQ ID NO: 1) and the ER-export signal FCYENEV (SEQ ID NO: 2) which both increased surface expression. Proper membrane localization of the eTags (GroupVII of FIG. 5) (FIG. 7A-C) was achieved, though it was found that the use of GFP produced lower levels of surface expression, which was enhanced when both motifs were added to the construct. The superiority of red fluorescent protein may be due to the greater hydrophobicity of red moieties versus green moieties. Surface expressed eTags were found to efficiently bind their substrate validating the effectives of this system for targeting of imaging molecules (FIG. 8).

### Example 5: MFS-Agents Membrane Permeability

Membrane permeability is a great concern for these MFS molecules. Firstly, increasing permeability of the agents through the plasma membrane abrogates the need for the fusion proteins containing the enzyme to be expressed on the cell surface. Second, in order for systemically administered MFS molecules to reach protected tissues such as the brain and testes, highly hydrophobic barriers (blood-brain, and blood-testes barriers) must be crossed. The greater the hydrophobicity of the molecules, the greater the permeability and subsequently the greater the amounts of molecules that can reach the brain.

The addition of the fluorescent moieties to the MFS molecules already improves the molecules permeability. The fluorescent moieties are already hydrophobic (the red more so than the green) and though the molecules easily dissolve in water due to the hydrophilic portions of the molecules, the fluorescent moieties still increased membrane permeability. Those molecules with multiple copies of the fluorescent moieties (groups VI and VII of FIG. 5) were even more membrane permeable.

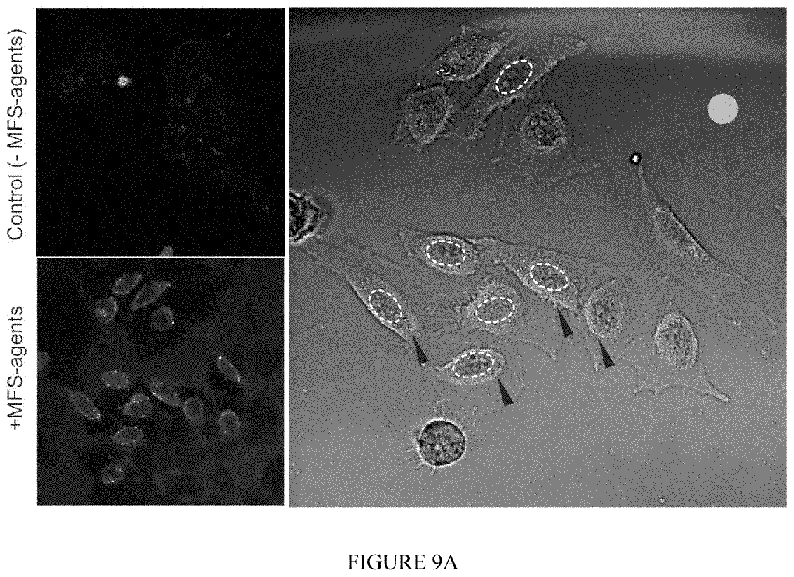

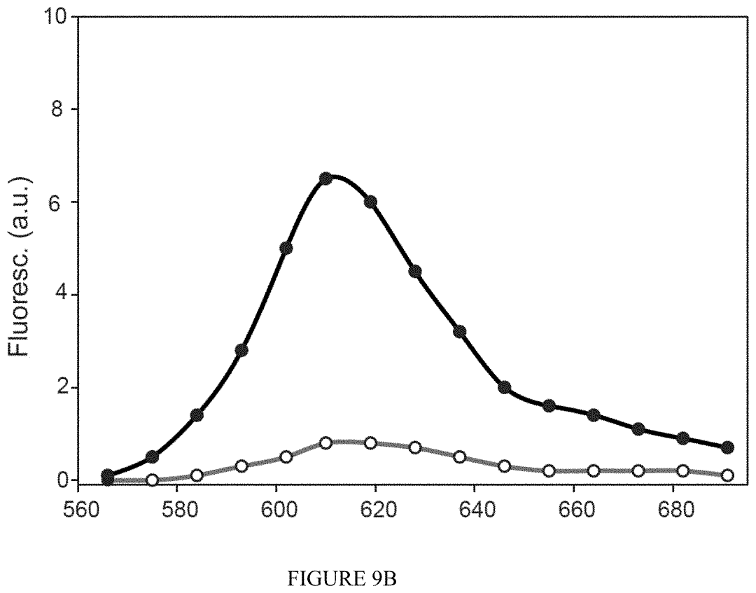

To even further increase membrane permeability the hydrophobic linkers of the molecule were extended, thus obtaining a larger hydrophobic surface. This, along with incorporation of silicone-containing derivatives of Rhodamine, further increased the hydrophobicity and hence, membrane permeability. These highly red molecules were administered to neuronal cells grown in culture. After incubation the cells were thoroughly washed and imaged to determine if the MFS molecules penetrated the plasma membrane. Indeed, a strong red signal, well above background (and controls where the MFS agents were not added) could be seen (FIG. 9A). Quantification of this signal confirmed a greater than 6-fold increase over non-labeled cells (FIG. 9B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A hybrid molecule comprising at least one contrast agent and at least one substrate of a self-labeling enzyme.

2. The hybrid molecule of claim 1, further comprising at least one fluorescent moiety.

3. The hybrid molecule of claim 1, wherein said at least one contrast agent is selected from: (i) a radiocontrast agent; (ii) contrast agent selected from the group consisting of a T1-class and a T2-class MRI contrast agent; (iii) an iodine based contrast agent; and (iv) a contrast agent comprising a metal selected from the group consisting of: a superparamagnetic metal, a paramagnetic metal, a diamagnetic metal, a ferromagnetic metal, or any combination thereof.

4. (canceled)

5. (canceled)

6. (canceled)

7. The hybrid molecule of claim 3, wherein any one of (i) said at least one paramagnetic metal is selected from the group consisting of Barium (Ba), Tantalum (Ta), Tungsten (W), Dysprosium (Dy), Platinium (Pt), Gadolinium (Gd), and Manganese (Mn); (ii) said at least one diamagnetic metal is selected from the group consisting of Bismuth (Bi) and Gold (Au); and (iii) said ferromagnetic metal is iron (Fe).

8. (canceled)

9. (canceled)

10. The hybrid molecule of claim 1, wherein said self-labeling enzyme is capable of covalently binding to said substrate.

11. The hybrid molecule of claim 10, wherein said self-labeling enzyme is selected from the group consisting of: SNAP, CLIP and Halo, optionally wherein said substrate is selected from the group consisting of: O⁶-benzylguanine derivatives, O²-benzylcytosine derivatives, and chloroalkene derivatives.

12. (canceled)

13. The hybrid molecule of claim 2, wherein said fluorescent moiety is capable of emitting UV, visible, or near infrared light, optionally wherein said fluorescent moiety is selected from the group consisting of: green fluorescent protein (GFP) and Tomato.

14. (canceled)

15. The hybrid molecule of claim 1, wherein said molecule has the general Formula:
(R)ₙ—(F)ₘ—(S)ₚ,
wherein:
R represents said contrast agent;
F represents said fluorescent moiety, and
S represents said substrate,
wherein n and p are each, independently, an integer from 1 to 5 and m is an integer from 0-5.

16. (canceled)

17. A composition comprising the hybrid molecule of claim 1, and a pharmaceutically acceptable carrier, excipient or adjuvant.

18. The composition of claim 17, comprising at least a first and a second hybrid molecule, wherein said substrate of said first hybrid molecule differs from said substrate of said second hybrid molecule, and at least one of said contrast agent and said fluorescent moiety of said first hybrid molecule differs from said contrast agent and said fluorescent moiety of said second hybrid molecule.

19. A cell-specific imaging method, the method comprising:
contacting one or more cells with a composition of claim 17, wherein at least one cell of said one or more cells expresses one or more self-labeling enzymes capable of binding to said substrate,
thereby imaging a specific cell.

20. The method of claim 19, wherein said one or more cells express said one or more self-labeling enzymes on said at least one cell's surface.

21. The method of claim 19, wherein said one or more cells are in the form of a tissue, optionally wherein said at least one cell is selected from a cancerous cell, a neuronal cell, and an immune cell.

22. The method of claim 19, wherein said contacting is performed ex-vivo or in-vivo.

23. (canceled)

24. The method of claim 19, comprising a preliminary step of expressing within said at least one cell said self-labeling enzymes capable of binding to said substrate.

25. The method of claim 24, wherein said expressing comprises introducing into said at least one specific cell one or more nucleic acid molecules comprising a polynucleotide sequence encoding said one or more self-labeling enzymes.

26. The method of claim 25, wherein said polynucleotide sequence encoding said one or more self-labeling enzymes is operatively linked to a cell-specific promoter.

27. The method of claim 19, further comprising applying a magnetic field, X-radiation, UV-vis light or any combination thereof, to said contacted cells.

28. The method of claim 27, further comprising measuring or detecting said contrast agent, said fluorescent moiety or both.

29. A kit comprising:
(a) at least one hybrid molecule of claim 1; and
(b) one or more nucleic acid constructs comprising a polynucleotide sequence encoding one or more self-labeling enzymes, optionally wherein said polynucleotide sequence encoding one or more self-labeling enzyme is operatively linked to a cell-specific promoter.

30. (canceled)
